(19) United States
(12) Patent Application Publication
(10) Pub. No.: US 2024/0398885 A1
(43) Pub. Date: Dec. 5, 2024

(54) FULL LIGHT REPAIR

(71) Applicant: Coty Inc., New York, NY (US)

(72) Inventors: Olivier Doucet, Roquebrune Cap Martin (FR); Cécile Chamayou-Robert, Nice (FR); Muriel Pujos, Nice (FR)

(21) Appl. No.: 18/694,262

(22) PCT Filed: Dec. 8, 2022
(86) PCT No.: PCT/US2022/081185

§ 371 Date: Mar. 21, 2024

(30) Foreign Application Priority Data
Dec. 10, 2021 (FR) FR2113318

(51) Int. Cl. A61K 36/31; A61K 8/44; A61K 8/9789; A61K 8/99; A61K 9/127; A61K 31/198; A61K 35/68; A61K 35/74; A61K 38/47; A61K 38/51; A61P 17/00; A61P 39/06; A61Q 19/08

(52) U.S. Cl. CPC A61K 36/31; A61K 8/44; A61K 8/9789; A61K 8/99; A61K 9/127; A61K 31/198; A61K 35/68; A61K 35/74; A61K 38/47; A61K 38/51; A61P 17/00; A61P 39/06; A61Q 19/08; C12Y 302/02017; C12Y 401/99003; C12Y 402/99018; A61K 2800/5922

(57) ABSTRACT

Provided herein is a method to treat skin damage due to light comprising topically administering to a subject in need thereof a composition comprising a sufficient amount of Arabidopsis thaliana extract, micrococcus lysate and plankton extract to simultaneously treat the damage caused by skin exposure to UVA, UVB, infrared light, visible light or a combination thereof.

### CLAIM OF PRIORITY

This patent application claims the benefit of priority to French Application Serial No. 2113318, filed Dec. 10, 2021, which is incorporated by reference herein in its entirety.

### BACKGROUND

Human skin is constantly exposed to sunlight. Solar radiation reaching the earth surface includes Ultra-Violet (UV) radiation (290-400 nm), visible light (400-760 nm) and infrared (IR) radiation (760 nm-1 mm). Each of these wavelengths generate specific DNA damage, such as oxidative lesions (as 8-oxoguanine) and cyclobutane pyrimidine dimers (CPD). DNA damage contributes to premature skin ageing.

### SUMMARY

The compositions and methods provided herein repair DNA damage induced by UVB, UVA, visible light, including blue-light, and infra-red. It has now been found that the application, especially topical application, of Arabidopsis thaliana extract, micrococcus lysate and plankton extract alone and/or in combination provides for minimizing and/or preventing, including reversing, the molecular, micro- and macro-manifestation of skin aging, especially skin aging due to light induced-UV, VIS, IR, UV-induced/light, and/or photodamage.

### BRIEF DESCRIPTION OF FIGURES

FIG. 1 provides a graph depicting the capacity of the compositions disclosed herein to enhance DNA-repair of oxidative DNA damage and CPD damage induced by solar radiations (improvement in DNA repair). It includes Ultra-violet, Blue-Light as part of Visible Light and Infra-Red wavelength.

FIG. 2 provides a graph depicting the capacity of the compositions disclosed herein to enhance DNA-repair of oxidative DNA damage and CPD damage induced by solar radiations (increase in DNA repair rate). It includes Ultra-violet, Blue-Light as part of Visible Light and Infra-Red wavelength.

FIGS. 3(a) and 3(b) provide graphs depicting the capacity of examples of the compositions disclosed herein comprising creatine to enhance DNA-repair of light induced CPD damage. UVB and Visible light induced CPD damage was repaired.

FIGS. 4(a), 4(b) and 4(c) provide graphs depicting the capacity of examples of the compositions disclosed herein comprising creatine to enhance DNA-repair of light induced oxidative DNA damage. It includes Ultra-violet, Blue-Light as part of Visible Light and Infra-Red wavelength. UVA, Visible and IR induced oxidative lesions were repaired.

FIG. 5 provides a graph showing the DNA repair dose-response models of each extract (enzyme).

### DETAILED DESCRIPTION

Human skin is constantly exposed to sunlight. Solar radiation reaching the earth surface includes Ultra-Violet (UV) radiation (290-400 nm), visible light (400-760 nm) and infrared (IR) radiation (760 nm-1 mm). It has been largely admitted that exposure of human skin to solar radiations results in a wide range of adverse effects, including photoallergic reactions, photodermatoses, autoimmune diseases, accelerated aging and cancers. UVB (280-315 nm), which are constituted by the most energetic wavelengths reaching the earth surface, penetrate into the superficial layers of the skin. They interact with both intracellular chromophores and DNA-bases to induce oxidative stress and direct DNA-damage. Due to their lower energetic wavelengths, UVA (315-400 nm) have been shown to be less deleterious than UVB for DNA. However, they penetrate deeper in the skin layers and produce oxidative stress, leading to both oxidized DNA-bases and DNA-lesions. Visible light (400-760 nm), which accounts for approximately 50% of the total solar spectrum, has long been considered safe for human skin. But it has been demonstrated that it could significantly contribute to the total DNA-lesions induced by solar exposure. Infrared light is considered far less genotoxic than the other wavelengths since it is weakly absorbed by intracellular chromophores, however it has been shown to contribute to skin-aging by modulating MMP and collagen gene expression. There is a need in the art to address the issues of light exposure to skin.

Embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the present invention is defined by the appended claims and their equivalents.

In addition, it is to be understood that the phraseology or terminology employed herein, and not otherwise defined, is for the purpose of description only and not of limitation.

All percentages and ratios used herein are by weight of the total composition and all measurements made are at 25° C., unless otherwise designated.

All numeric ranges are inclusive of narrower ranges; delineated upper and lower range limits are interchangeable to create further ranges not explicitly delineated. The number of significant digits conveys neither limitation on the indicated amounts nor on the accuracy of the measurements.

In this document, the terms “a” or “an” are used to include one or more than one and the term “or” is used to refer to a nonexclusive “or” unless otherwise indicated. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The compositions of the present invention can comprise, consist essentially of, or consist of, the essential as well as optional ingredients and components described herein. As used herein, “consisting essentially of” means that the composition or component may include additional ingredients, but only if the additional ingredients do not materially alter the basic and novel characteristics of the claimed compositions or methods.

The term “topical application,” as used herein, means to apply or spread the compositions of the present invention onto the surface of the skin and also hair, nails and other mammalian, such as human, keratinous tissue.

The phrase “dermatologically-acceptable” or “cosmetically acceptable” as used herein, means that the compositions or components thereof so described are suitable for use in contact with human skin without undue toxicity, incompatibility, instability, allergic response, and the like.

A “dermatologically acceptable vehicle” refers to a material that acts as a diluent, dispersant or carrier for the stated actives and is recognized in the industry as acceptable or suitable for use, including long term use, in skin contact and, to the extent appropriate or applicable, has been approved or is otherwise approvable by a regulatory agency of a government or governmental body or is listed in the U.S. Pharmacopoeia or other generally recognized pharmacopoeia for use on humans.

The term “improves” or “improved” is used to convey that the present invention changes either the characteristics and/or the physical attributes of the tissue to which it is being provided, applied or administered and/or influences the biomolecular activities and/or processes in the skin in a way that counteracts, directly or indirectly, the detrimental effects of UV exposure, air pollutant exposure, and/or skin aging.

The term “inhibiting” generally refers to the ability to prevent or delay the onset of a given event, process, action or reaction, or symptom or manifestation of a condition or disorder.

The term “optional” or “optionally” means that the subsequently described subject, event or circumstance may or may not occur, and that the description includes instances where the event occurs and instances where it does not and/or when the subject is present and when it is not present.

The terms “effective amount” and “skin enhancing effective amount” refer to the amount of the specified compound or composition that when applied to the skin is able to affect the desired effect whether on a molecular level, as evidenced by changes in the level or concentration of targeted materials, and/or on a macro level whereby visual or microscopic changes in the tissue are evident. Skin enhancing refers to both the ability to inhibit, prevent and/or preclude damage as well as the ability to reverse existing damage.

“Skin care actives” as used herein, means substances that when applied to the skin, provide a benefit or improvement to the skin. It is to be understood that skin care actives are useful not only for application to skin, but also to hair, nails and other mammalian keratinous tissue.

“Situs” means the location where the composition is applied. Non-limiting examples of a situs include mammalian, such as human, keratinous tissue.

As used herein, the term “purified” and like terms relate to an enrichment of a molecule or compound relative to other components associated with the molecule or compound. The term “purified” does not necessarily indicate that complete purity of the particular molecule has been achieved during the process.

The term “substantially pure” describes a compound which has been separated from components which accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound is also substantially purified when it is essentially free of associated components or when it is separated from the native contaminants which accompany it in its natural state.

The terms “comprises,” “comprising,” and the like can have the meaning ascribed to them in U.S. Patent Law and can mean “includes,” “including” and the like. As used herein, “including” or “includes” or the like means including, without limitation.

### Composition

The topical compositions of the subject invention, including but not limited to lotion, serum, oil, essence, mask, night cream, stick, eye cream, color cosmetic or cream comprising a combination of Arabidopsis thaliana extract, micrococcus lysate, and plankton extract, in for example a 1:1:1 ratio, and optionally including creatine. The inventors have found that the inclusion of the extracts in the ratio of 1:1:1 advantageously enhances DNA repair.

The compositions described herein repair cyclobutane pyrimidine dimers (CPD) and repair oxidative lesions, caused by UV, visible light, including blue light, and Infra-red exposure, thereby resulting in a composition that enhances DNA repair of damaged skin cells. The compositions can be used to enhance DNA repair of damaged skin cells where the damage is caused by exposure to the full spectrum of light, for example wavelengths from 290 nm to 1,1050 nm.

Arabidopsis thaliana Extract

The compositions of the present invention can include Arabidopsis thaliana extract. Arabidopsis thaliana extract is an anti-aging skin care ingredient. Arabidopsis thaliana prompts a liposome delivery system (involving the repair enzyme known as 8-oxo-guanine DNA glycosylase-1 (OGG1; enzyme able to repair oxidative damage on DNA, both in nucleus and mitochondria)), which identifies DNA damage in your skin, and proceeds to assist the body's natural process in restoration by beginning the cellular repair process, and by transporting enzymes into the skin. Basically, it recognizes the damage in your skin, and then initiates the body's natural way of repairing it, speeding up the process of restoring your skin. In some aspects, Arabidopsis thaliana extract is present in the liposome from 0.025% to 2.5% (w/w), including 0.025% to 2.0%, 0.025% to 1.5% 0.030% to 2.5%, 0.040% to 2.5%, 0.025% to 1.0%, including 0.025%, 0.030%, 0.040%, 0.050%, 0.060%, 0.070%, 0.080%, 0.090%, 0.10%, 0.20%, 0.30%, 0.40%, 0.50%, 0.60%, 0.70%, 0.80%, 0.90%, 1.0%, 1.25%, 1.5%, 1.75%, 2.0%, 2.25% or 2.5%. In some aspects, the Arabidopsis thaliana extract is present in the formula from 0.00005% to 0.02 (w/w), including 0.00006% to 0.02% (w/w), 0.00007% to 0.02% (w/w), 0.00008% to 0.02% (w/w), 0.00009% to 0.02% (w/w), 0.0001% to 0.02% (w/w), 0.0002% to 0.02% (w/w), 0.0003% to 0.02% (w/w), 0.0004% to 0.02% (w/w), 0.0005% to 0.02% (w/w), 0.00005% to 0.01% (w/w), 0.0005% to 0.009% (w/w), 0.0005% to 0.008% (w/w), 0.00005% to 0.06% (w/w), 0.00005% to 0.007% (w/w), 0.00006% to 0.0008% (w/w), 0.00007% to 0.007% (w/w), 0.00008% to 0.006% (w/w), 0.00009% to 0.007% (w/w) or 0.0001% to 0.005% (w/w).

### Micrococcus Lysate

UV-endonuclease (a natural DNA repair enzyme); an enzyme from an extract of ocean bacteria. It's enclosed in a tiny package of fat (called a liposome) that aids in the deliver of the enzyme into the skin. It's an end product of the lysis of various species of Micrococcus (such as UV-endonuclease enzymes encapsulated in liposomes). In some aspects, micrococcus lysate is present in the liposome from 0.025% to 2.5% % (w/w), in the liposome, including 0.03% to 2.5%, 0.04% to 2.5%, 0.05% to 2.5%, 0.025% to 2.4%, 0.025% to 2.3%, 0.025% to 2.2%, including 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2% and 2.5%. In some aspects, the micrococcus lysate is present in the formula from 0.00005% to 0.02 (w/w), including 0.00006% to 0.02% (w/w), 0.00007% to 0.02% (w/w), 0.00008% to 0.02% (w/w), 0.00009% to 0.02% (w/w), 0.0001% to 0.02% (w/w), 0.0002% to 0.02% (w/w), 0.0003% to 0.02% (w/w), 0.0004% to 0.02% (w/w), 0.0005% to 0.02% (w/w), 0.00005% to 0.06% (w/w), 0.00005% to 0.07% (w/w), 0.00005% to 0.08% (w/w), 0.00005% to 0.009% (w/w), 0.00005% to 0.01% (w/w), 0.00006% to 0.06% (w/w), 0.00007% to 0.07% (w/w), 0.00008% to 0.08% (w/w), 0.00009% to 0.09% (w/w) or 0.0001% to 0.01% (w/w).

### Plankton Extract

Plankton extract contains photolyase which uses the energy of visible light to reverse the DNA damage, cyclobutane pyrimidine dimers (CPD), induced by UVB. Plankton extract can be encapsulated in a liposome delivery system. In some aspects, plankton extract is present from 0.025% to 2.5% % (w/w), in the liposome, including 0.1% to 2.5%, 0.5% to 2.5%, 1% to 2.5%, 0.025% to 2.4%, 0.025% to 2.3%, 0.025% to 2.0%, including 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2% and 2.5%. In some aspects, the plankton extract is present in the formula from 0.00005% to 0.02% (w/w), including 0.00006% to 0.02% (w/w), 0.00007% to 0.02% (w/w), 0.00008% to 0.02% (w/w), 0.00009% to 0.02% (w/w), 0.0001% to 0.02% (w/w), 0.0002% to 0.02% (w/w), 0.0003% to 0.02% (w/w), 0.0004% to 0.02% (w/w), 0.0005% to 0.02% (w/w), 0.00005% to 0.01% (w/w), 0.0005% to 0.009% (w/w), 0.00005% to 0.008% (w/w), 0.00005% to 0.007% (w/w), 0.00005% to 0.006% (w/w), 0.00006% to 0.006% (w/w), 0.00007% to 0.007% (w/w), 0.00008% to 0.008% (w/w), 0.00009% to 0.009% (w/w) or 0.00001% to 0.01% (w/w).

### Creatine

The compositions of the present invention advantageously include creatine. Creatine is an organic compound with the formula (H₂N)(HN)CN(CH₃)CH₂CO₂H. Creatine exists in various modifications (tautomers) in solution. Creatine is found in vertebrates where it facilitates recycling of adenosine triphosphate (ATP), primarily in muscle and brain tissue. Recycling is achieved by converting adenosine diphosphate (ADP) back to ATP via donation of phosphate groups. In some aspects, creatine is present in the formula from 0.001% to 10% (w/w), including 0.002% to 9.0%, 0.003% to 8%, 0.004% to 7%, 0.02% to 5.0%, 0.05% to 4.0%, 0.10% to 3.0%, 0.20% to 2.0%, 0.04% to 1.5%, including 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.10%, 0.20%, 0.30%, 0.40%, 0.50%, 0.60%, 0.70%, 0.80%, 0.90%, 1.00%, 1.50%, 2.00%, 2.50%, 3.00%, 3.50%, 4.00%, 4.50%, 5.00%, 5.50%, 6.00%, 6.50%, 7.00%, 7.50%, 8.00%, 8.50%, 9.00% 9.50% or 10.00 (w/w). In one embodiment, creatine can be included in one or more liposomes.

### Carrier/Delivery System

The actives can be alone on in combination with carrier/delivery system. The compositions of the present invention comprise from about 0.000001 to about 99% of an acceptable carrier/delivery system within which the Arabidopsis thaliana extract, micrococcus lysate, plankton extract, creatine or a combination thereof and additional materials are incorporated to enable these components to be applied/delivered topically at an appropriate concentration. The carrier can thus act as a diluent, dispersant, solvent, or the like for the particulate material which ensures that it can be applied to and distributed evenly over the selected target at an appropriate concentration. The actives can also be encapsulated in one or more encapsulants, such as liposomes or other vehicles including biodegradable polymers/nanocapsules, hydrogels, cyclodextrins, poly(lactic-co-glycolic)acid (PLGA) or PLCA microspheres.

The carrier may contain one or more dermatologically acceptable solid, semi-solid or liquid fillers, diluents, solvents, extenders and the like. The carrier may be solid, semi-solid or liquid.

The carrier can be substantially liquid. The carrier can itself be inert or it can possess dermatological benefits of its own. Concentrations of the carrier can vary with the carrier selected and the intended concentrations of the components.

Suitable carriers include conventional or otherwise known carriers that are dermatologically acceptable. The carrier should also be physically and chemically compatible with the essential components described herein, and should not unduly impair stability, efficacy or other use benefits associated with the compositions of the present invention. Components of the compositions of this invention should be capable of being comingled in a manner such that there is no interaction which would substantially reduce the efficacy of the composition under ordinary use situations.

The type of carrier utilized herein depends on the type of product form desired for the composition. The topical compositions useful in the subject invention may be made into a wide variety of product forms such as are known in the art. These include, but are not limited to, lotions, creams, gels, sticks, sprays, ointments, oils, pastes, mousses and cosmetics (e.g., solid, semi-solid, or liquid make-up, including foundations, eye-makeup, pigmented or non-pigmented lip treatments, e.g., lipsticks, and the like). These product forms may comprise several types of carriers including, but not limited to, solutions, aerosols, emulsions, gels, solids, and oils.

Carriers can contain a dermatologically acceptable diluent. As used herein, “diluent” includes materials in which the material can be dispersed, dissolved, or otherwise incorporated, such as a lipophilic diluent/carrier. Solutions according to the subject invention typically include a dermatologically acceptable diluent. Solutions useful in the subject invention can contain from about 0.000001 to about 99% of the diluent.

Aerosols according to the subject invention can be formed by adding a propellant to a solution such as described above. Exemplary propellants include chloro-fluorinated lower molecular weight hydrocarbons. Additional propellants that are useful herein are described in Sagarin, Cosmetics Science and Technology, 2nd Edition, Vol. 2, pp. 443-465 (1972), incorporated herein by reference. Aerosols are typically applied to as a spray-on product.

Carriers can comprise an emulsion comprising a hydrophilic phase comprising a hydrophilic component, e.g., water or other hydrophilic diluent, and a hydrophobic phase comprising a hydrophobic component, e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the composition ingredients. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% of the dispersed hydrophobic phase and from about 1% to about 98% of the continuous hydrophilic phase; water-in-oil emulsions typically comprise from about 1% to about 98% of the dispersed hydrophilic phase and from about 1% to about 50% of the continuous hydrophobic phase. The emulsion may also comprise a gel network, such as described in “Application of Emulsion Stability Theories to Mobile and Semisolid Oil-in-Water Emulsions”, Cosmetics and Toiletries, vol. 101, November 1986, pp. 73-92, which is incorporated by reference herein.

The topical compositions of the subject invention, including but not limited to lotion, serum, oil, essence, mask, night cream, stick, eye cream, color cosmetic or cream, may comprise a dermatologically acceptable emollient. Such compositions can contain from about 2% to about 50% of the emollient. Emollients tend to lubricate the skin, increase the smoothness and suppleness of the skin, prevent or relieve dryness of the skin, and/or protect the skin. Emollients are typically water-immiscible, oily or waxy materials. A wide variety of suitable emollients are known and may be used herein. Sagarin, Cosmetics, Science and Technology 2nd Edition, Vol. 1, pp. 32-43 (1972), incorporated herein by reference, contains numerous examples of materials suitable as an emollient.

Lotions and creams according to the present invention generally comprise a solution carrier system and one or more emollients. Lotions typically comprise from about 1% to about 20%, such from about 5% to about 10%, of emollient; from about 50% to about 90%, including from about 60% to about 80%, water. A cream typically comprises from about 5% to about 50%, including from about 10% to about 20%, of emollient; and from about 45% to about 85%, including from about 50% to about 75%, water.

Ointments of the present invention may comprise a simple carrier base of animal or vegetable oils or semi-solid hydrocarbons (oleaginous); absorption ointment bases which absorb water to form emulsions; or water-soluble carriers, e.g., a water-soluble solution carrier. Ointments may further comprise a thickening agent, such as described in Sagarin, Cosmetics, Science and Technology, 2nd Edition, Vol. 1, pp. 72-73 (1972), incorporated herein by reference, and/or an emollient. For example, an ointment may comprise from about 2% to about 10% of an emollient; and from about 0.1% to about 2% of a thickening agent.

Compositions of this invention useful for cleansing (“cleansers”) are formulated with a suitable carrier, e.g., as described above, and preferably contain one or more dermatologically acceptable surfactants in an amount which is safe and effective for cleansing. For example, such compositions contain from about 1% to about 90%, more preferably from about 5% to about 10%, of a dermatologically acceptable surfactant. The surfactant can be selected from anionic, cationic, nonionic, zwitterionic, amphoteric and ampholytic surfactants, as well as mixtures of these surfactants. Such surfactants are well known to those skilled in the detergency art. Nonlimiting examples of possible surfactants include isoceteth-20, sodium methyl cocoyl taurate, sodium methyl oleoyl taurate, sodium lauryl sulfate, and betaines such as described herein. See U.S. Pat. No. 4,800,197, to Kowcz et al., issued Jan. 24, 1989, which is incorporated herein by reference in its entirety, for exemplary surfactants useful herein. Examples of a broad variety of additional surfactants useful herein are described in Mccutcheon's Detergents and Emulsifiers, North American Edition (1986), published by Allured Publishing Corporation, which is incorporated herein by reference in its entirety. The cleansing compositions can optionally contain, at their art-established levels, other materials which are conventionally used in cleansing compositions.

The physical form of the cleansing compositions can be, for example, formulated as bars, liquids, shampoos, bath gels, hair conditioners, hair tonics, pastes, or mousses. Bars are most preferred since this is the form of cleansing agent most commonly used to wash the skin. Preferred rinse-off cleansing compositions, such as shampoos, include a delivery system adequate to deposit sufficient levels of actives on the skin and scalp. A delivery system can involve the use of insoluble complexes. For a more complete disclosure of such delivery systems, see U.S. Pat. No. 4,835,148, Barford et al., issued May 30, 1989, incorporated herein by reference in its entirety.

The composition may also take the form of a cosmetic composition that may be applied to mammalian keratinous tissue, including human skin. The cosmetic compositions may take various forms. For example, some non-limiting examples of forms include solutions, suspensions, lotions, oils, creams, gels, toners, sticks, pencils, ointments, pastes, foams, powders, mousses, shaving creams, wipes, strips, patches, electrically powered patches, wound dressing and adhesive bandages, hydrogels, film-forming products, facial and skin masks, cosmetics (e.g., foundations, eye liners, eye shadows), and the like.

For example, the cosmetic composition may comprise from 1% to 95% by weight of water. The cosmetic composition may comprise from 1% to 95% by weight of one or more oils. Oils may be used to solubilize, disperse, or carry materials that are not suitable for water or water soluble solvents. Suitable oils include silicones (such as dimethicone), hydrocarbons, esters, amides, ethers, and mixtures thereof. When the cosmetic composition is in the form of an emulsion, oils are carriers typically associated with the oil phase. The cosmetic composition may be in the form of a water-in-oil emulsion, an oil-in-water emulsion, or a water-in-silicone emulsion such that the cosmetic composition may include water, a silicone, oil, and combinations thereof. The cosmetic compositions may include an emulsifier. An emulsifier is particularly suitable when the cosmetic composition is in the form of an emulsion or if immiscible materials are being combined. The cosmetic composition may comprise from 0.05%, 0.1%, 0.2%, 0.3%, 0.5%, or 1% to 20%, 10%, 5%, 3%, 2%, or 1% emulsifier. Emulsifiers may be nonionic, anionic, zwitterionic, or cationic. Structuring agents may be used to increase viscosity, thicken, solidify, or provide solid or crystalline structure to the cosmetic composition. Structuring agents are typically grouped based on solubility, dispersibility, and phase compatibility. Examples of aqueous or water structuring agents include, but are not limited to, polymeric agents, natural or synthetic gums, polysaccharides, and the like. The cosmetic compositions may comprise from 0.0001%, 0.001%, 0.01%, 0.05%, 0.1%, 0.5%, 1%, 2%, 3%, 5% to 25%, 20%, 10%, 7%, 5%, 4%, or 2%, by weight of the cosmetic composition, of one or more structuring agents. The cosmetic compositions may optionally contain one or more UV actives. As used herein, “UV active” includes both sunscreen agents and physical sunblocks. Suitable UV actives may be organic or inorganic. Examples of some suitable UV actives are listed in the functional category of “Sunscreen Agents” in the Personal Care Product Council's International Cosmetic Ingredient Dictionary and Handbook, Thirteenth Edition, 2010. The cosmetic compositions may be generally prepared by conventional methods such as those known in the art of making cosmetic compositions. Such methods typically involve mixing of ingredients in one or more steps to a relatively uniform state, with or without heating, cooling, application of vacuum, and the like. Typically, emulsions are prepared by first mixing the aqueous phase materials separately from the fatty phase materials and then combining the two phases as appropriate to yield the desired continuous phase. The cosmetic compositions are preferably prepared such as to optimize stability (physical stability, chemical stability, photostability, etc.) and/or delivery of active materials. The cosmetic composition may be provided in a package sized to store a sufficient amount of the cosmetic composition for a treatment period. The size, shape, and design of the package may vary widely.

Optionally, the present topical composition may include one or more of the invention compositions may optionally comprise other active and inactive ingredients, including, but not limited to, excipients, fillers, emulsifying agents, antioxidants, surfactants, film formers, chelating agents, gelling agents, thickeners, emollients, humectants, moisturizers, vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, colorants, staining agents, depigmenting agents, hypopigmenting agents, preservatives, stabilizers, pharmaceutical agents, photostabilizing agents, and mixtures thereof. In addition to the foregoing, the personal care products of the invention may contain any other compound for the treatment of skin disorders.

The cosmetic compositions disclosed herein may be applied to one or more skin surfaces and/or one or more mammalian, such as human, keratinous tissue surfaces as part of a user's daily routine or regimen. Additionally, or alternatively, the cosmetic compositions herein may be used on an “as needed” basis. In some examples, an effective amount of the cosmetic composition may be applied to the target portion of the keratinous tissue or skin. In some examples, the cosmetic composition may be provided in a package with written instructions detailing the application regimen.

### Uses Thereof

Skin aging is a complex process that involves metabolic and physiologic changes. Besides chronological aging and its impact on human skin and its physiological processes and functions, a number of environmental factors and exposures also have a marked impact upon skin aging, or at least the appearance and manifestation of those consequences associated with or arising from skin aging. Perhaps no environmental factor or exposure is more detrimental to the skin than the exposure of skin to UV radiation and visible light exposure (including but not limited to, UVA, UVB, infrared light, visible light, including blue light) which causes a number of diverse biological effects, including sunburn (inflammation), induction of skin cancer (melanoma), premature skin aging, and alteration in cutaneous immune cells (immunosuppression), all of which lead to damage, including permanent damage, of the skin cells. Unfortunately, skin cell damage due to UV radiation and visible light exposure is induced by several mechanisms such as UV-induced immunosuppression, UV-induced DNA damage and accumulation of DNA damaged products, such that efforts to protect the skin, for example, by application of sunscreen, application of moisturizers, post-sunburn treatments and the like, while effective in some respects, are ineffective in others. Rather, for comprehensive photo-protection, especially against premature skin aging, photo-allergies, immune-suppression and skin cancer, it is believed necessary to reverse or reduce UV- and light-induced changes in the skin.

Provided herein are Arabidopsis Thaliana extract, micrococcus lysate and plankton extract. Combined, these agents repair cyclobutane pyrimidine dimers (CPD) and repair oxidative lesions, caused by UV, visible light and Infra-red exposure, thereby resulting in a composition that enhances DNA repair of damaged skin cells.

The compositions of the present invention are useful for topical application, such as a cosmetic and to improve the aesthetic appearance of skin, including visible and/or tactile discontinuities in skin. Such discontinuities may be induced or caused by internal and/or external factors and include the signs of skin aging and sun or other light damage.

The phrase “improve the aesthetic appearance of skin” includes prophylactically and/or therapeutically improving a skin condition, including visible and/or tactile discontinuities in skin. As used herein, prophylactic use includes delaying, minimizing and/or preventing visible and/or tactile discontinuities in skin. As used herein, therapeutically improving a skin condition includes ameliorating, e.g., diminishing, minimizing and/or effacing, discontinuities in skin (improving skin appearance and/or feel).

The compositions of the present invention are useful for preventing or treating or improving signs of skin aging and sun or other light induced damage. Improving the signs of skin aging and/or improving the aesthetic appearance of skin includes prophylactically and/or therapeutically improving one or more of such signs (similarly, improving a given sign of skin aging, e.g., lines, wrinkles or pores, includes prophylactically and/or therapeutically improving that sign). As used herein, prophylactic use includes delaying, minimizing and/or preventing signs of skin aging. As used herein, therapeutically improving such signs includes ameliorating, e.g., diminishing, minimizing and/or effacing signs of skin aging.

“Signs of skin aging” include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles, including both fine superficial wrinkles (fine lines) and coarse deep wrinkles, skin lines, crevices, bumps, large pores (e.g., associated with adnexal structures such as sweat gland ducts, sebaceous glands, or hair follicles), scaliness, flakiness and/or other forms of skin unevenness or roughness, loss of skin elasticity (loss and/or inactivation of functional skin elastin), sagging (including puffiness in the eye area and jowls), loss of skin firmness, loss of skin tightness, loss of skin density, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

It is to be understood that the present invention is not to be limited to improvement of the above mentioned “signs of skin aging” which arise due to mechanisms associated with skin aging but is intended to include improvement of said signs irrespective of the mechanism of origin. As used herein, “improving a skin condition” is intended to include improving of such signs irrespective of the mechanism of origin.

The present invention is especially useful for improving visible and/or tactile discontinuities in mammalian, such as human, skin texture, including texture discontinuities associated with skin aging. As used herein, therapeutically improving such discontinuities includes ameliorating, e.g., diminishing, minimizing and/or effacing visible and/or tactile discontinuities in the texture of mammalian, human skin, to thereby provide improved skin appearance and/or feel, e.g., a smoother, more even appearance and/or feel. Such visible and/or tactile discontinuities in skin texture include crevices, bumps, pores, fine lines, wrinkles, scales, flakes and/or other forms of textural unevenness or roughness associated with skin aging. For example, the length, depth, and/or other dimension of lines and/or wrinkles are decreased, the apparent diameter of pores decreases.

The present invention is also useful for prophylactic uses that include preventing, inhibiting or delaying visible and/or tactile discontinuities in mammalian, such as human, skin texture, including texture discontinuities associated with skin aging. As used herein, prophylactic use includes delaying, minimizing and/or preventing visible and/or tactile discontinuities in the texture of mammalian skin, to thereby provide improved skin appearance and/or feel, e.g., a smoother, more even appearance and/or feel.

The compositions described herein can used to treat and/or prevent a variety of skin conditions and/or disorders. Skin conditions/disorders can include aging, signs of aging, acne, dermatitis, rosacea, psoriasis and/or wounds. Signs of aging can include wrinkles, fine lines, wizened skin, skin laxity associated with collagen loss or destruction, loss or reduction in skin integrity, lack of skin elasticity, lack of skin tone, thinned skin, sagging skin, skin suffering from degradation of collagen fibers, flaccid skin, and/or skin suffering from internal degradation. The conditions/disorders can include inflammation, broken veins, redness, blotchiness, puffy eyes or dark circles, or skin pigmentation disorders. The compositions provided herein are also useful for promoting youthful looking skin, for promoting evenness of skin tone by reducing skin redness or inflammation or UV induced skin redness, blotchiness or inflammation, for promoting skin regeneration to produce more homogenous, for promoting/increasing fibronectin levels in skin cells, firmer, more toned and more elastic skin, for promoting cell longevity, for promoting skin brightness, for promoting skin texture and tone uniformity and/or for reducing the appearance of skin pigmentation and/or skin darkening, for treating or preventing ulcerated areas or areas of cutaneous stress or damage brought about by exposure to UV or exposure to an irritant product, for enhancing skin desquamation, improving skin hydration, improving skin luster and brightness, or for treating or reducing acne or other skin blemishes.

The compositions disclosed herein may be applied to one or more topical surfaces and/or one or more mammalian, such as human, keratinous tissue surfaces as part of a user's daily routine or regimen. Additionally, or alternatively, the compositions herein may be used on an “as needed” basis and used for as intended for the given consumer product. The composition may be applied to any article, such as a textile, or any absorbent article.

Exemplary compositions of this invention include:

One aspect provides a sunscreen formula comprising, in addition to active:
Water
Diisopropyl Adipate
Decyl Cocoate
Glycerin
Bis-Ethylhexyloxyphenol Methoxyphenyl Triazine
Isoamyl P-Methoxycinnamate Tocopherol
Dimethylmethoxy Chromanol
Ethylhexyl Triazone
Butyl Methoxydibenzoylmethane
Ethylhexyl Salicylate
Dicaprylyl Carbonate Tocopherol
Poly C10-30 Alkyl Acrylate
Polyglyceryl-6 Stearate Polyglyceryl-6 Behenate
Glyceryl Stearate Se
Carbomer
Disodium Edta
Xanthan Gum
Caprylyloxyphenylamino Dimethyltetrahydro Benzothiazine Carboxylic Acid
Citric Acid
Mica
Titanium Dioxide
Bambusa Arundinacea Stem Extract
Silica
Boron Nitride
Fragrance

Another aspect provides an after-sun formula comprising, in addition to active:
Water
Butylene Glycol
Butyrospermum Parkii (Shea) Butter
C13-14 Isoparaffin Laureth-7
Caprylic/Capric Triglyceride
Cetearyl Alcohol Ceteareth-20
Cetyl Palmitate
Chlorphenesin
Dimethylmethoxy Chromanol
Glycerin
Hydrogenated Polyisobutene Tocopherol
Polyacrylamide Water
Xanthan Gum
Panthenol
Mauritia Flexuosa Fruit Oil
Sodium Lactate Methylsilanol
Hydrolyzed Citrus Aurantium Dulcis Fruit
Bisabolol
Fragrance

The following examples are intended to further illustrate certain embodiments of the invention and are not intended to limit the scope of the invention in any way.

### EXAMPLES

### Example 1

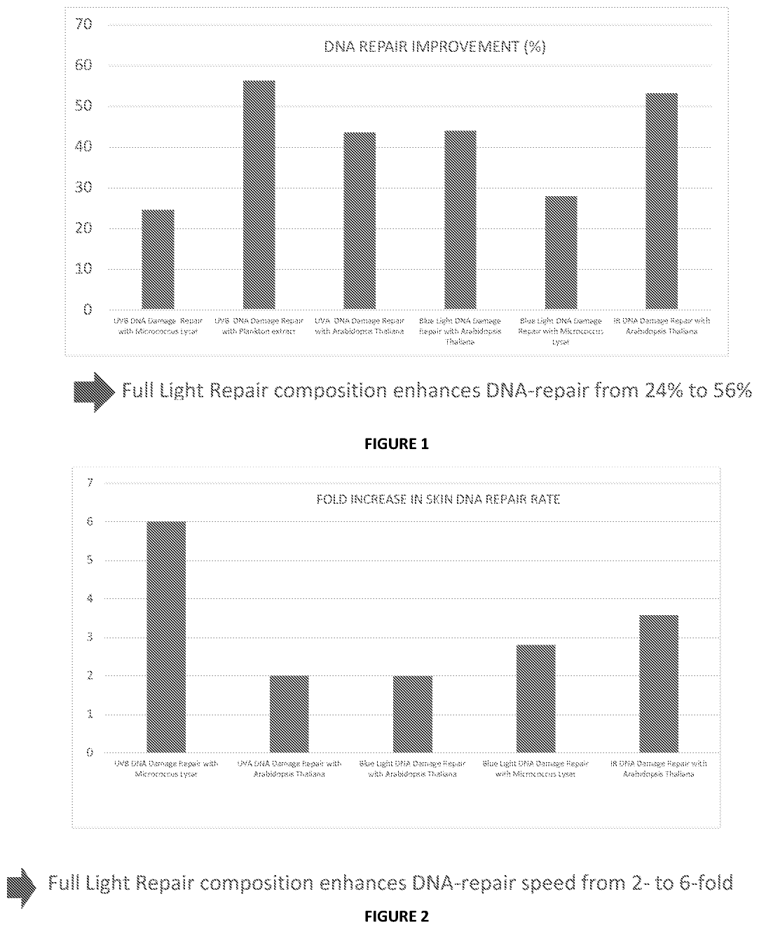

Experiments were carried out as discussed in Chamayou-Robert et al. Photodermatol Photoimmunol Photomed. 2021; 00:1-7. Briefly, experiments were performed on normal human keratinocytes. Cells were exposed to either UVB (280-315 nm), UVA (315-400 nm), blue light (400-500 nm), or infra-red (760 nm-1 mm) for a time to induce DNA damage. Cells were then treated with the composition indicated in the FIGS. 1 and 2 and DNA damage was analyzed by using the comet assay (Wischermann et al. Mutat Res. 2007; 630:122-128; Jean et al. Photochem Photobiol. 2007; 74 (3): 417-423; Azqueta et al. Mutagenesis. 2013; 28:271-277; Chamayou-Robert et al. Photoderm Photoimmun. 2021; 00:1-7) with the use of specific enzymes, formamidopyrimidine DNA glycosylase (FPG) or T4 endonuclease V in order to detect oxidized DNA-bases and cyclobutane pyrimidine dimers (CPD) respectively.

### Example 2

Experiments were carried out to demonstrate the boosting effect of creatine on the DNA repair of oxidative DNA damage and CPD (cyclobutane pyrimidine dimers) damage induced by light.

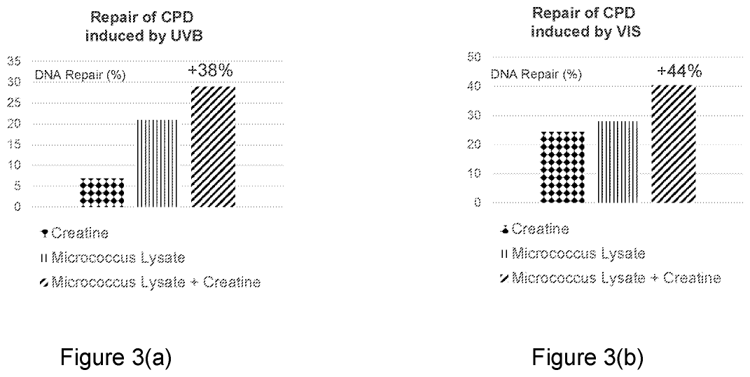

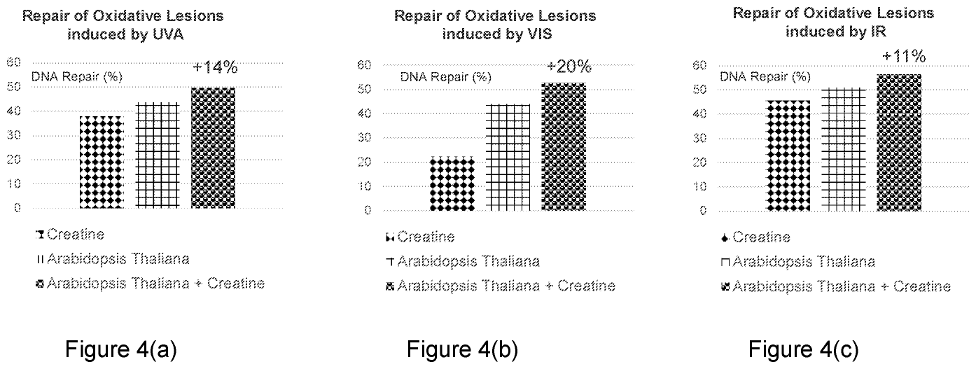

Experiments were carried out using the protocol discussed in Chamayou-Robert et al. Photodermatol Photoimmunol Photomed. 2021; 00:1-7. Briefly, experiments were performed on normal human keratinocytes. Cells were exposed to either UVB (280-315 nm), UVA (315-400 nm), visible light, blue light (400-500 nm), or infra-red (760 nm-1 mm) for a time to induce DNA damage. Cells were then treated with the compositions indicated in FIGS. 3(a), 3(b) and 4(a), 4(b) and 4(c), respectively and DNA damage was analyzed by using the comet assay (Wischermann et al. Mutat Res. 2007; 630:122-128; Jean et al. Photochem Photobiol. 2007; 74 (3): 417-423; Azqueta et al. Mutagenesis. 2013; 28:271-277; Chamayou-Robert et al. Photoderm Photoimmun. 2021; 00:1-7) with the use of specific enzymes, formamidopyrimidine DNA glycosylase (FPG) or T4 endonuclease V in order to detect oxidized DNA-bases and cyclobutane pyrimidine dimers (CPD) respectively.

Compositions comprising creatine in an amount of 0.5% w/w, Micrococcus Lysate in an amount of 0.0005% w/w and Arabidopsis Thaliana extract in an amount of 0.0005% w/w were tested in this example.

The results demonstrated the surprising technical effect that creatine has a boosting effect on the DNA repair of oxidative DNA damage and CPD damage induced by light, in particular UVA, UVB, IR and visible light. The inclusion of creatine in the compositions was found to boost the amount of DNA repair. As shown in FIGS. 3(a), 3(b) and 4(a), 4(b) and 4(c), creatine has a boosting effect on DNA repair of light induced DNA damage.

With reference to FIGS. 3(a) and 3(b), the boosting effect of creatine on the percentage DNA repair of oxidative DNA damage and CPD damage was demonstrated when creatine was included in a composition comprising micrococcus lysate. For example, as shown in FIG. 3(a), DNA repair of CPD damage induced by UVB was boosted by +38% when creatine was used in combination with micrococcus lysate and as shown in FIG. 3(b) DNA repair of CPD damage induced by visible light was boosted by +44% when creatine was used in combination with micrococcus lysate.

With reference to FIGS. 4(a) to 4(c), the inventors have identified that creatine demonstrated a surprising technical effect of boosting the DNA repair of oxidative damage induced by UVA, visible and IR light. For example as shown in FIG. 4(a) DNA repair of oxidative lesions induced by UVA was boosted by +14% when creatine was used in combination with Arabidopsis thaliana extract. FIG. 4(b) demonstrates that DNA repair of oxidative lesions induced by visible light was boosted by +20% when creatine was used in combination with Arabidopsis thaliana extract. FIG. 4(c) demonstrates that DNA repair of oxidative lesions induced by Infra-Red light was boosted by +11% when creatine was used in combination with Arabidopsis thaliana extract.

### Example 3

The inventors have found that the inclusion of Arabidopsis thaliana extract, micrococcus lysate, and plankton extract in the ratio of 1:1:1 advantageously enhances DNA repair.

Dose response modelling was carried out to determine the optimum ratios of the ingredients. DNA repair performance of each extract containing enzyme was assessed. Dose response curves were modelled for each enzyme and each type of stress (UVB, UVA, Visible Light (VL), IR) by using polynomial models. From these equations, the respective DNA repair was calculated for the different ratios of enzymes.

$$( \frac{UVB - median}{range} ) * 0.5 + ( \frac{UVA - median}{range} ) * 0.4 + ( \frac{VL - median}{range} ) * 0.3 + ( \frac{IR - median}{range} ) * 0.3$$

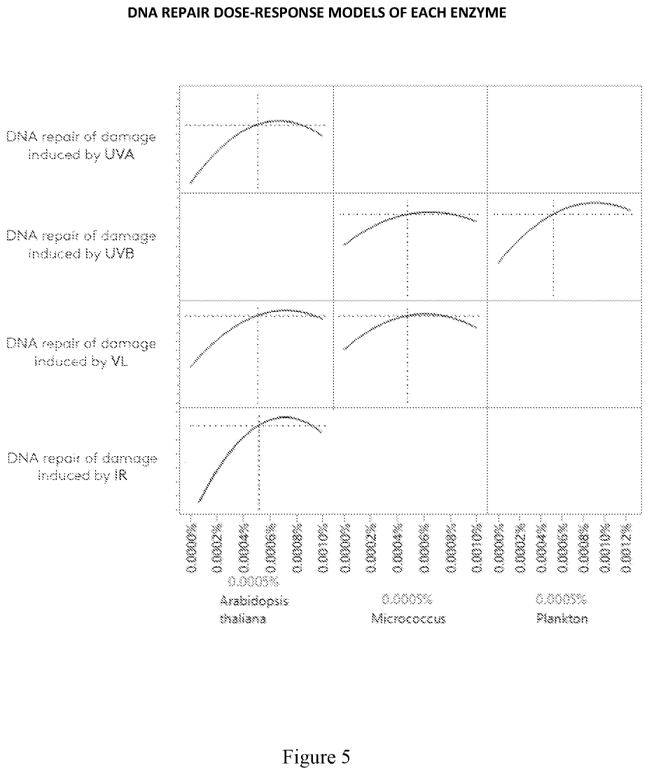

FIG. 5 shows the DNA repair dose-response models of each extract containing enzyme. The results demonstrated that a ratio of 1:1:1 of Arabidopsis thaliana extract, micrococcus lysate, and plankton extract was the optimum ratio for DNA repair performance.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

1. A composition comprising about 0.00005% to 0.02% (w/w) of Arabidopsis thaliana extract; about 0.00005% to 0.02% (w/w) micrococcus lysate, about 0.00005% to 0.02% (w/w) of plankton extract, and creatine.

2-3. (canceled)

4. The composition of claim 1, wherein the DNA damage is due to exposure to blue light, wherein the DNA damage is oxidative DNA-damage and/or cyclobutane pyrimidine dimers (CPD) DNA-damage, wherein the blue light damage is detected by a comet assay with the use of formamidopyrimidine DNA glycosylase (FPG) or T4 endonuclease V in order to detect oxidized DNA-bases and cyclobutane pyrimidine dimers (CPD) respectively.

5. The composition of claim 1, wherein the composition comprises Arabidopsis thaliana extract, micrococcus lysate and plankton extract in a ratio of 1:1:1.

6. The composition of claim 1, wherein the composition comprises creatine in an amount from 0.001% to 10% (w/w).

7. The composition of claim 1, wherein the composition comprises creatine in an amount from 0.05% to 4.0% (w/w).

8. The composition of claim 1, wherein the composition comprises about 0.0001% to 0.02% (w/w) of Arabidopsis thaliana extract.

9. The composition of claim 1, wherein the composition comprises about 0.0001% to 0.02% (w/w) micrococcus lysate.

10. The composition of claim 1, wherein the composition comprises about 0.0001% to 0.02% (w/w) of plankton extract.

11. The composition of claim 1, wherein the DNA damage is cyclobutene-pyrimidine-dimers (CPD) and/or oxidative lesions.

12. The composition of claim 1, wherein the Arabidopsis extract comprises oxo-guanine glycosylase-1 (OGG1), the micrococcus lysate comprises UV-endonuclease and the plankton extract comprises photolyase.

13. The composition of claim 1, wherein the composition further comprises a liposome.

14. The composition of claim 13, wherein the liposome is lecithin.

15. The composition of claim 14, wherein the liposome comprises phosphatidyl ethanolamine, phosphatidyl choline, cholesteryl hemisuccinate or combination thereof.

16. A composition comprising plankton extract (photolyase) in an amount from about 0.00005% to 0.02% (w/w), Arabidopsis thaliana extract (oxo-guanine glycosylase-1 (OGG-1)) in an amount from about 0.00005% to 0.02% (w/w) and micrococcus lysate (UV-endonuclease) in an amount from about 0.00005% to 0.02% (w/w), and creatine.

17. A method to treat skin damage due to light comprising topically administering to a subject in need thereof a composition comprising about 0.00005% to 0.02% (w/w) Arabidopsis thaliana extract, about 0.00005% to 0.02% (w/w) micrococcus lysate, about 0.00005% to 0.02% (w/w) plankton extract, and creatine, to simultaneously treat the damage caused by skin exposure to UVA, UVB, infrared radiation, visible light or a combination thereof.

18. A method to simultaneously repair DNA damage in skin cells caused by skin exposure to visible light, UVB, UVA, and/or infrared radiation comprising topically applying to skin cells a composition comprising about 0.00005% to 0.02% (w/w) Arabidopsis thaliana extract, about 0.00005% to 0.02% (w/w) micrococcus lysate, about 0.00005% to 0.02% (w/w) plankton extract, and creatine.

19. A method to reduce premature skin ageing comprising topically applying to skin cells a composition comprising about 0.00005% to 0.02% (w/w) Arabidopsis thaliana extract, about 0.00005% to 0.02% (w/w) micrococcus lysate, about 0.00005% to 0.02% (w/w) plankton extract, and creatine.

20. The method of claim 17, wherein the DNA damage is due to exposure to blue light, wherein the DNA damage is oxidative DNA-damage and/or cyclobutane pyrimidine dimers (CPD) DNA-damage, wherein the blue light damage is detected by a comet assay with the use of formamidopyrimidine DNA glycosylase (FPG) or T4 endonuclease V in order to detect oxidized DNA-bases and cyclobutane pyrimidine dimers (CPD) respectively.

21. The method of claim 17, wherein the composition comprises Arabidopsis thaliana extract, micrococcus lysate and plankton extract in a ratio of 1:1:1.

22. The method of claim 17, wherein the composition further comprises creatine in an amount from 0.001% to 10% (w/w).

23. The method of claim 17, wherein the composition comprises creatine in an amount from 0.05% to 4.0% (w/w).

24. The method of claim 17, wherein the composition comprises about 0.0001% to 0.02% (w/w) of Arabidopsis thaliana extract.

25. The method of claim 17, wherein the composition comprises about 0.0001% to 0.02% (w/w) micrococcus lysate.

26. The method of claim 17, wherein the composition comprises about 0.0001% to 0.02% (w/w) of plankton extract.

27. The method of claim 17, wherein the DNA damage is cyclobutene-pyrimidine-dimers (CPD) and/or oxidative lesions.

28. The method of claim 17, wherein the Arabidopsis extract comprises oxo-guanine glycosylase-1 (OGG1), the micrococcus lysate comprises UV-endonuclease and the plankton extract comprises photolyase.

29. The method of claim 17, wherein the composition further comprises a liposome.

30-31. (canceled)
